(19) United States
(12) Patent Application Publication
(10) Pub. No.: US 2021/0393668 A1
(43) Pub. Date: Dec. 23, 2021

(54) USE OF OLIGONUCLEOTIDES FOR THE TREATMENT OF TUMORS

(71) Applicant: HAEMES Verwaltungsgesellshaft mbH, Hamburg (DE)

(72) Inventor: Heinrich Maria SCHULTE, Hamburg (DE)

(21) Appl. No.: 17/425,019

(22) PCT Filed: Jan. 21, 2020
(86) PCT No.: PCT/EP2020/051338

§ 371 Date: Jul. 22, 2021

(30) Foreign Application Priority Data
Jan. 23, 2019 (DE) 10 2019 000 490.6

(51) Int. Cl. A61K 31/7088; A61K 47/69; A61K 47/36; A61K 47/64; A61K 9/06

(52) U.S. Cl. CPC A61K 31/7088; A61K 47/6903; A61K 9/06; A61K 47/6435; A61K 47/36

(57) ABSTRACT

In a method of treating a patient having a solid tumor, the solid tumor is completely or partially resected or ablated and then a therapeutically-effective amount of an oligonucleotide that kills tumor cells is applied or administered in a body cavity created by the resection or ablation in order to kill tumor cells remaining in, or in the surroundings of, a tumor bed and/or to suppress metastases. Thus, the oligonucleotide counteracts the development of recurrences of the solid tumor or new metastases. The oligonucleotide may act, e.g., in a pleiotropic manner in at least one type of tumor cell.

The invention relates to the use of oligonucleotides for the specific therapy of tumor diseases at various stages directly during surgical intervention.

Short single- or double-stranded oligonucleotides referred to as oligonucleotides may be synthesized by a chemical process established in the prior art (Herdewijn 2005; Surhone et al. 2010). Currently, DNA oligonucleotides up to a length of about 200 bases or base pairs may be synthesized efficiently, wherein, for many technical reasons, DNA may be synthesized more easily and in longer fragments compared to RNA. Technically, longer syntheses are possible, but enzymatic or in vivo preparations are simpler and more efficient. However, in the future it is expected that oligonucleotides of increasing length will also be synthesized. As of now, machines are used in the synthesis process, which perform the repetitive steps of the synthesis automatically.

One advantage of chemically synthesized oligonucleotides lies in their modifiability. Modifications may comprise, for example, non-natural nucleotide building blocks such as inosine or natural modifications such as cytosine methylation. Furthermore, nucleotides may be modified at the base residue, the sugar residue, or the phosphate residue, as present in embodiments of the present invention. Modifications include, for example, substitutions with alkyl, alkoxy, amino, deaza, halogen, hydroxyl, thiol groups or combinations thereof. Nucleotides may also be replaced with analogs of higher stability, for example, a ribonucleotide may be replaced with a deoxyribonucleotide or the 2′ OH group of its sugar moiety may be replaced with 2′ amino groups, 2′ O-methyl groups, 2′ methoxyethyl groups, or a 2′-O, 4′-C methylene bridge. Examples of a purine or pyrimidine analog of nucleotides include xanthine, hypoxanthine, azapurine, methylnthioadenine, 7-deaza-adenosine, and O- or N-modified nucleotides. The phosphate residue of the nucleotide may be modified by replacing one or more oxygen atoms of the phosphate group with nitrogen or with sulfur (phosphorothioates). Further modifications include Locked Nucleic Acids LNA (WO9914226 A2), Unlocked Nucleic Acids UNA, 2′OMe-methoxy- and 2′F fluoro- modifications. The 3′ and 5′ ends of a single strand also make possible the addition of further molecular residues such as simple or glycosylated peptides and proteins as well as lipids, chitosan and other chitosan derivatives, polymers or dyes. Modifications of this type may be linked to the oligonucleotides directly or via spacers, for example one or more glycine residues. These modifications are developed with a variety of objectives, for example for influencing the stability of the oligonucleotides or the melting temperature of a double strand, for changing the affinity to other molecules or surfaces or the biological availability or activity in vivo. Modifications are also used, for example, for improving the function of the oligonucleotide, their stability or their transfer properties, or for controlling their localization or targeting. In the following, the term “oligonucleotides” includes all of these modifications.

Many types of oligonucleotides occur naturally in humans and animals or generally in living beings or viruses or are derived there from. They have very different functions such as the regulation of the expression of proteins, the interference with the expression or also the communication between cells. They may be present as a double strand, a double strand with single-strand overhangs, a hairpin, cyclically or as a single strand. Examples of naturally occurring oligonucleotides are microRNA (miRNA) or long noncoding RNA (InRNA); examples of naturally derived or artificial oligonucleotides are short interfering RNA (siRNA), short activating RNA (saRNA), CRISPR-Cas oligonucleotides or antisense-DNA, of which some classes are suitable as therapeutically active substances. Antisense DNA (Mipomersen™, Kastle Theraputics, Inc., Chicago, Ill., USA) for the treatment of homozygous, hereditary hypercholesterolemia and siRNA (Patisaran™, Alnylam, Inc., Cambridge, Mass., USA) for the treatment of polyneuropathy in hereditary, transthyretin mediated amyloidosis, are approved for first uses. Both medicaments are injected systemically.

The term “tumor” in the narrower sense is understood to refer to a benign or malignant neoplasm (neoplasia) of body tissues resulting from a dysregulation of cell growth (DocCheck, https://flexikon.doccheck.com/de/Tumor). Colloquially, malignant tumors are referred to as cancer. To this day, there is no cure for many forms of malignant, in particular solid, tumors. For example, among tumors having a very short survival time after diagnosis, are those of the pancreas, of the adrenal glands, of the mesothelium, of the brain and of the lungs, although great efforts have been undertaken in recent decades in order to improve or develop diagnoses, individual therapies and treatment methods for these tumors. Examples include the surgical removal of the tumor, medicinal treatment or radiation, and also in various combinations. Newer or experimental treatment methods include specific gene-therapy methods or cell therapies, including, in particular, chimeric antigen receptor-bearing T cells (CAR-T).

Despite better diagnostic technologies and despite the enormous therapeutic efforts and the early detection of tumors, improvements achieved so far concerning treatment and survival are very limited. For many of these tumors, patients are cured only in exceptional cases. Many therapies, however, at least slow disease progression and extend life expectancy of the patient. Depending on the type of tumor, the mean lifetime gained is between years and only a few months. There is a considerable need for new therapies, particularly for those tumors, in the case of which patients under treatment so far have gained hardly any additional life expectancy.

Many solid tumors are treated early and in a first step using surgery. A complete removal of the primary tumor is not always possible, however. Even when the tumor mass appears to be completely removed, it is very likely that tumor cells are still present in the tumor bed or in the margins of the surgical cavity, from which a recurrence may arise. These tumor cells are not detectable even when using sensitive pathological procedures. Metastases occurring at a short distance from the primary tumor are also likely (colloquially, seeding metastases). In some types of tumors, a complete removal is almost impossible, for example in advanced glioblastomas. Glioblastomas grow in an infiltrating manner and then often affect indispensable brain regions. In some types of tumors, even the entire affected organ is surgically removed, such as the adrenal gland in adrenocortical carcinoma. In this case, medicinal substitution of organ function is necessary and also possible. Even with radical resection, recurrences are common and almost always fatal for this tumor. Despite these limitations, surgical removal of the tumor often is the essential component of an efficient tumor treatment for which there is no substitute.

Tumor resection often is accompanied with an adjuvant or neoadjuvant therapy. Medicaments and radiation are then used in a neoadjuvant manner, i.e., before the surgery, if the tumor mass of the primary tumor and the risk of surgery both are to be reduced, as for example in the case of large tumors or tumors that are difficult to reach.

Medicaments and radiation therapy are intended to inhibit in an adjuvant manner, i.e., after the operation, the occurrence of recurrences and to act on distant metastases which are not surgically accessible or whose existence is only suspected. The radiation therapy, which is used postoperatively in most cases, serves in particular to prevent a recurrence-development or to treat known local metastases, since it is employed almost only locally nowadays. Medicaments are utilized systemically in most cases, especially if metastases are suspected but can not be detected.

In the above-mentioned applications, medicaments may be chemotherapeutics, which have a cytotoxic effect predominantly on proliferating cells, or targeted therapeutics such as antibodies, or antibodies loaded with cytotoxins, which attack tumor cells via tumor-specific receptors. In the case of metastases, since the receptor composition often is not known, targeted therapeutics are rarely used as adjuvants.

For inoperable tumors, only radiation and medicaments are available. In order to reduce the undesirable effects of medicinal therapies, nowadays they are also applied locally. This is often accomplished via an injection directly into the tumor or into surrounding tissue if an expected spread of tumor cells is to be avoided during the direct injection.

Less often, medicaments are administered locally during surgery. It is expected that distant metastases are not reached any better than adjuvant systemic applications. For glioblastoma, which often is only partially resectable, such treatment strategies are however pursued more often and with the goal that the non-resectable tumor mass is thereby attacked.

To this end, chemotherapies are used, which are introduced into the surgical cavity after the surgery. In the case of glioblastoma, a local administration is also considered, since many medicaments do not cross the blood-brain barrier—even though the blood-brain barrier may not be completely intact in the case of a glioblastoma. Here, polymeric carriers are also used, which are intended to make possible an easier application and a longer availability and significantly higher dosages of the medicament by serving as a deposit having a slow release of the medicament stored therein, without the occurrence of toxic systemic effects.

Gliadel™, which was approved in the USA in 1999, is an example of a biodegradable, disc-shaped carrier having a diameter of 14 mm and a thickness of 1 mm, wherein the carrier is provided with 7.7 mg of the cytostatic carmustine (also: bis-chloroethyl-nitroso-urea, abbreviated BCNU); up to eight specimens are inserted into the patient's surgical cavity by the surgeon towards the end of the operation after removal of the tumor tissue. Clinical studies showed a prolongation of the median lifespan by approximately 2 months for both patients diagnosed for the first time and patients with recurrences (Hart et al. 2008), but the significance of the studies is questioned (Chowdhary et al. 2015). The wafers are detectable up to 232 days after the surgery and are therefore present longer than the assumed proliferation cycle of the tumor cells.

The Gliadel™ carriers, however, are large and brittle. Contact between carrier and surgical cavity is therefore circumscribed. The usage instructions, however, do not provide for the carriers to be broken up into smaller fragments because an increased surgery risk is feared. An entire series of other carrier principles have therefore been developed, which have reached different stages of clinical development, including microbeads (Paclimer™, Guildford Pharmaceuticals) having a diameter of 53 mm, which have been provided with paclitaxel (taxol) for intraperitoneal administration in recurrent ovarian cancer. They also were used in a modified form for the treatment of glioma (Li et al. 2003).

Another form of nanoparticles which expand at acidic pH (<5) has been developed. Supposedly, these nanoparticles release their substances in the acidic environment of the endosome. These expanding particles were also provided with paclitaxel for use in the treatment of lung cancer (Griset et al. 2009), mesothelioma (Schulz et al. 2011) and peritoneal carcinomatosis (Colson et al. 2011). The particles were administered intravenously or intraperitoneally after resection of the primary tumor in mouse models. Apparently, these particles have not yet been tested clinically.

Chitosan-based hydrogels: Chitosan is obtained by deacetylating chitin, the material from which insect and crustacean shells are made essentially. Due to its excellent biodegradability, chitosan has found its way into biomedical applications for some time, for example as a wound healing material (Kim et al., 2008). Paclitaxel-loaded, chitosan-based hydrogels have been suggested for treating wound healing margins after tumor resection. Therefore, the combination was injected in vivo a few days after tumor inoculation in order to simulate this situation (Ruel-Gariapy et al., 2004). Campthotecin was used in another study, but without the goal of application during resection (Berrada et al. 2005).

In the case of radio frequency or thermal ablation, micro rods provided with doxorubicin were injected as adjuvants. In the case of ablation, recurrences are most frequently found at the margin of the treatment zone and around blood vessels. In animal experiments, the rods were injected after ablation of the liver (Qian et al. 2003) as well as into a pre-punctured xenograft liver tumor (Weinberg et al., 2007). The formation of a fibrotic capsule around the rod was reduced by a simultaneous administration of dexomethasone, which was complexed with hydroxypropyl β-cydodextrin (Blanco et al., 2006). In another experiment, the rods were provided with 5-FU (Haaga et al., 2005).

Flexible films which adapt better to the surface of wound margins than rigid carriers are a further possible carrier, so that substances may diffuse through a larger area into the tissue. Poly(glycerol monostearate co-epsilon-caprolactone) films provided with paclitaxel were implanted after resection in a lung cancer model (Liu et al. 2010); in a similar model, with hydroxycamptothecin (Wolinski et al. −2, 2010) and once again with paclitaxel in a sarcoma-model (Liu et al., 2012).

The often limited success of these strategies led to further refinements of local administration, for example, the use of liposomes for cytostatics (Yang et al. 2016), the coupling of targeting molecules to nanoparticles or controlled release of the medicaments by various external stimuli such as magnetic fields or light (Rosenblum et al., 2018).

A novel, intraoperative form of application is called hyperthermic intraperitoneal chemotherapy (HIPEC) (Glehen et al. 2008), with which a high local concentration of cytostatics in the peritoneum and the absorption of the substance in the upper cell layers is to be achieved with reduced systemic toxicity. The additional hyperthermia is believed to increase the therapeutic potential of the cytostatics used by improving tissue penetration. However, the hyperthermia also produces its own direct cytotoxic effect (Ceelen et al. 2010).

For the application of HIPEC, several drainage lines are placed in different areas of the abdomen at the end of surgery. A roller pump system having a heat exchanger is used and the temperature progression is controlled. The target temperature is 42-43° C., and the perfusion time is between 30 and 120 minutes, depending on the protocol used. The thus-far, most-used cytostatic drug is mitomycin C. The standard medicaments used in standard systemic therapy, oxaliplatin and irinotecan, are being increasingly employed in HIPEC (Piso et al. 2011).

Pressure aerosol chemotherapy (Pressurized IntraPeritoneal Aerosol Chemotherapy, PIPAC) is another form of application. The application of an aerosol under pressure results in a particularly effective distribution of medicaments in body cavities such as the abdomen or the chest. During PIPAC, medicaments (such as cisplatin, doxorubicin, oxaliplatin, paclitaxel) are used. The local administration is effected directly at the tumor site so that the otherwise existing pharmacological limitations of intraperitoneal chemotherapy, such as poor distribution within the body cavity and low diffusion into the tissue, are eliminated. The local dose of the cytostatic may be reduced by a factor of 10 without losing tumor efficacy. Thus, dose-dependent local toxicity of intraperitoneal chemotherapy is better controlled, and organ toxicity as well as systemic side effects of the therapy are significantly reduced.

In PIPAC with a 12 mmHg CO2, a capnoperitoneum, i.e., an overpressure, is applied, and two balloon trocars are inserted through the abdominal wall. Then, a micropump is inserted into the abdomen and connected to a high-pressure contrast medium injector. In a typical protocol, doxorubicin (1.5 mg/m² body surface area (BSA) in 50 ml 0.9% NaCl solution) and cisplatin (7.5 mg/m²BSA in 150 ml 0.9% NaCl solution) are applied successively via the micropump and injector as a pressure chemotherapy aerosol. Injection parameters are set at a flow rate of 30 ml/min and a maximum inlet pressure of 200 psi. The micropump generates a polydisperse aerosol having a droplet size between 6 to 11 p.m. This small size ensures that the dispersed droplets remain within the gas for an extended period of time, for example over 30 minutes. The therapeutic capnoperitoneum is kept at a temperature of 37° C. for 30 minutes. The chemotherapy aerosol is then excreted into the air disposal system via a closed line. Finally, the trocars are pulled (Reymond et al. 2014).

An adjuvant gene therapy strategy, Sitimagene ceradenovec (Cerepro), has been published (EP 1 135 513 B2). Here, after tumor resection, an adenovirus is injected into the tissue adjacent to the surgical cavity, wherein the virus has a functional thymidine kinase gene, for example of the herpes-simplex virus. In transfected cells, this gene causes the production of thymidine kinase. When ganciclovir is applied after surgery and reaches the transfected cells, it is preferably phosphorylated in these cells into an active substance that interferes with gene synthesis. Owing to this procedure, the effect of ganciclovir is confined locally to the region around the surgical cavity. Clinical test results, however, did not result in an approval of this therapy; the negative recommendation of the Committee for Medicinal Products for Human Use (CHMP), based on the evaluation by the Committee for Advanced Therapies (CAT) of the European Medicines Agency (EMA), was justified by the low observed effectiveness and the significant risk of serious side effects (EMA web site for withdrawn applications for approval: Cerepro). Later on, this approach was developed further by additionally administering the cytostatic temezolomide, which is part of the standard therapy for glioblastomas, due to an observed synergistic effect (EP 2 665 489 B1).

Furthermore, a gene therapy is proposed for mesothelioma and ovarian cancer (WO 2015/002861 A1), wherein an adenovirus loaded with a gene for human interferon alpha 2b is used. Here, the adjuvant application is not explicitly provided.

Recently, attempts have been made to use antibodies labeled with dyes, in particular, fluorescent dyes, during tumor resection in order to visualize tumor-positive resection margins during surgery. In a breast cancer model, the dye IRDye800CW was coupled to the antibodies bevacizumab, cetuximab, panitumumab, trastuzumab and tocilizumab and their local distribution was investigated (Korb et al. 2014). In another study, a monoclonal antibody against PD-1 was labeled with the same dye and injected intraperitoneally after surgery as an adjuvant without dye (Du et al. 2017). Moreover, it has been proposed to use photoimmunotherapy, i.e., antibody-based photodynamic therapy, intraoperatively during tumor resection in the tumor bed in order to simultaneously visualize and fight remaining tumor cells (de Boer 2016).

In studies, however, antibodies were also used in an adjuvant manner in tumor treatment and in fact in the form of multiple injections systemically after the resection of the tumor. In the case of colon cancer, for example, adjuvant chemotherapy after surgery has become state of the art and is often used in medical practice. Here, 5-FU and oxaliplatin are used. In studies, the angiogenesis-inhibiting monoclonal antibody Avastin™ (bevacizumab) was instead systemically administered postoperatively. This antibody binds to the vascular endothelial growth factor (VEGF) and inhibits the angiogenesis signaling. After 36 months, however, no improvement was shown with regard to the risk of recurrence in the adjuvant antibody arm of the study. It is used with cetuximab, an EGFR-binding antibody in breast cancer therapy, In another study, this antibody was additionally added to the standard for adjuvant chemotherapy. As a result, the relapse risk for the antibody arm hardly changed; on the contrary, the relapse risk increased for some patient groups (De Gramont et al. 2011, Oyan B 2012). The antibodies are believed to accelerate the resistance development to chemotherapies. From preclinical studies, it is also known that their adjuvant use promotes the development of metastases. Furthermore, it cannot be excluded that new pro-survival paths are stimulated, which act in a resistance developing manner. It also appears that in combined therapies, an interference of the mechanisms of action also may take place (Huang et al. 2017)

Immune check point-targeted monoclonal antibodies (ICT mAbs), for example PD-1 antibodies, were administered intratumorally in first clinical tests. Compared to a systemic use, resistance to ICT mAbs appears to be reduced (Maraballe et al. 2017).

Several oligonucleotides already have been developed for the treatment of tumors. Usually, they are directed against specific cellular targets, for example, cell cycle proteins such as kinesin spindle protein (KSP), polo-like kinase 1 (PLK), protein kinase N3 (PKN3), ribonucleotide reductase (RRM2), or tenasion-2. Also included is a prodrug from WO 2010/102615, wherein a conjugated protease substrate inhibits the effectiveness of the siRNA until the substrate is cleaved by a protease.

In the case of oligonucleotides, however, current developments focus primarily on the formulation and the delivery as nanoparticles or gel (Kim et al. 2012), which are intended for the systemic or intraperitoneal application as an injection. siRNA may also be applied, for example, “naked” in saline, or complexed with polycations, cationic lipid/lipid transfection reagents or cationic peptides, as components of defined molecular conjugates (e.g., cholesterol-modified siRNA, TAT-DRBD/siRNA complexes), or as components of liposomes. For applications in regenerative medicine, however, the use of a matrix made of porous polyester urethane was tested as a subcutaneous implant, releasing siRNA-binding nanoparticles from a diblock copolymer (Nelson et al. 2013), thereby requiring two carrier systems, which means additional complexity. Simultaneous inclusion of cells and siRNA in alginate or collagen has also been described (Krebs et al. 2009) and has been proposed for applications in which the loaded alginate or collagen, present as a hydrogel, may be injected.

Local applications of oligonucleotides have been and are being developed as intratumoral injections for a variety of therapeutic approaches against tumors. Han et al. used chitosan particles formulated with siRNA for reducing in vivo expression of transglutaminase in breast cancer and melanoma (Hat et al. 2012). Hydrogels having a gelling temperature of 40° C. in combination with gold-containing nanoshells were injected intratumorally to induce a local effect of siRNA by using optical radiation (Strong et al., 2014). Polyethyleneimide-conjugated organophosphazenes also exhibit a thermal reaction and were injected intratumorally with siRNA against VEGF and Cyclin B1 (Kim et al. 2012). These approaches were not used perioperatively or in an adjuvant manner; the focus was solely on achieving a high local concentration and thus a high efficiency against the primary tumor, and on reducing the speed of diffusion of oligonucleotides, compared to small molecules, from the site of action. The same also applies to the study on ductal pancreatic adenocarcinoma (PDA), in which the expression of a mutated form of the Kirsten RAt Sarcoma virus (KRAS) gene was to be reduced (Khvalevsky et al. 2013). In this approach, a polylactide-co-glycolide-(PLGA-) matrix is used as well. Matrix elements were even sutured to the tumor in order to optimize efficiency against the primary tumor through a large surface area contact. Immunostimulatory RNA (CPG 1826, Coley Pharmaceuticals) was administered intratumorally in an adjuvant manner in an animal experiment; here, mice were treated with antibodies against OX40, CTLA4, GITR and FR4, but not operated. The authors reported an improved effect; however, this approach was not pursued clinically (Houot R et al. 2009).

Plasmids and oligonucleotides also have already been locally applied in order to promote wound healing, e.g., plasmids for platelet-derived growth factor (pPDGF) or vascular-endothelial growth factor (pVEGF) in poly(lactic-co-glycolic acid) PLGA nanoparticles (Tokatlian et al. 2014) or also prolyl hydroxylase domain 2 (PHD2)-siRNA in acellular dermal matrix as an implant (Vandegrift et al. 2015), or p53 (Nguyen et al. 2010). In these applications, however, the focus is on wound healing after an injury without any connection to tumors. A potential risk of these approaches lies in the fact that the target genes addressed for wound healing may constitute an increased risk for tumor formation. This conjecture is an obstacle for a use of these local approaches in tumor treatments.

According to the state-of-the-art, cytostatics in particular are used as adjuvants after tumor resection. In fact, locally applied cytostatics are expected to exhibit many advantages with respect to their systemic application:
the substances, which often are hydrophobic, reach the intended site of action within the vicinity of the resected tumor mass much more directly
the otherwise rapid breakdown of the small cytostatics-molecules is counteracted by their application via carriers from which the molecules are eluted over a longer period of time. As a result, more cell cycles of the proliferating cells may be covered by the medication.
adverse effects outside the tumor, which are dose-limiting for many cytostatics, are limited to the area around the surgical cavity and are significantly lower.

The reasons for the limited success of such adjuvant systems so far are not known. To date, Gliadel™, a combination of macroscopic carrier and carmustine as a cytostatic, is the only system approved in the United States and Europe. The approval extends to the adjuvant use in glioblastoma.

The advantages of local, adjuvant application expected for small molecules are not expected for macromolecules. Macromolecules are usually readily soluble and are excreted more slowly than small molecules. In addition, the achievable concentration gain is lower with the local application of macromolecules than with small molecules. The diffusion paths of macromolecules are also shorter compared to those of the small molecules, i.e., they do not penetrate far into the tumor bed and its surroundings. The depth of penetration becomes particularly small if a fibrotic capsule is formed around the carrier during the healing process of the surgical cavity.

In tumor resection, macromolecules are therefore used for other purposes. An elaborate approach was followed with Sitimagene ceradenovec, which approach involves the conversion of a prodrug injected systemically after surgery, which in turn is a small molecule. However, the conversion only occurs locally in cells transfected previously during surgery with a gene for herpes simplex thymidine kinase, which has a length of several hundred nucleotides. The gene is packaged in a replication-incompetent adenovirus, with which the cells are infected. The gene is expressed after transfection of the cells and causes the herpes simplex thymidine kinase to be produced, which in turn converts the Ganciclovir™ prodrug into the active form. The use of the small molecule Gangciclovir™ makes it possible to achieve a high local concentration.

Antibodies are employed in the field of surgery, however, primarily due to their possible use as markers in a fluorescence-labeled manner during the surgery. Here, antibodies are often used, which otherwise find usage in a systematic manner as tumor therapeutics. Their ability to bind to tumor-specific receptors makes it possible to label tumor cells-specifically with them. Upon excitation of the fluorescence, the tumor cells thus labeled light up and provide the surgeon with information about the spread of the tumor. Antibodies are injected locally in an intratumor manner for therapy, i.e., without tumor resection. They are applied only systemically in an adjuvant manner for tumor resection.

For adjuvant, local tumor therapy, there are primarily small molecules available that have so far shown only limited therapeutic success. There is therefore a need for improved adjuvant therapy in order to improve the chances of a cure after tumor resection.

### OBJECT OF THE INVENTION

The object underlying the invention therefore is to provide a simple form of adjuvant therapy, which is applied locally within a surgical cavity, which therapy kills the tumor cells, which remain after an at least partial tumor resection and which develop after the resection within the tumor bed and within its vicinity, or seeding metastases in the vicinity or surroundings of the primary tumor.

### SOLUTION ACCORDING TO THE INVENTION

This object is achieved in a surprising manner by the use of tumor-effective oligonucleotides which are applied directly in the tumor bed after the tumor resection. Here, the oligonucleotides are preferably applied together with resorbable, gel-like or elastic carriers or solid carriers such as gauze material or particles. Also, if the surgical cavity is too complex for other forms of application, a liquid formulation may be preferred in order to reach all potential locations, where tumor cells may be located. In turn, the liquid form may contain particulate or gel-like particulate carriers. In the case of a liquid formulation, the volume to be applied is preferably chosen such that it fills the surgical cavity by more than a half, or, in particular, completely. If drainage is required after the surgery, the liquid application form may also be applied as a perfusion several times or continuously for a longer period of time. Furthermore, an aerosolic application form may also be preferred, in which the formulated or non-formulated oligonucleotides are aerosolized from a liquid solution and introduced into the surgical cavity.

This effect is surprising because it is expected of oligonucleotides that they are less suitable for this application compared to other classes of molecules, in particular to small molecules, due to their charge, size, lability and their low rate of uptake into cells and due of their rapid degradation. Formulated oligonucleotides are sometimes significantly larger and diffuse even more slowly, rendering them even less suitable for this use according to expectations.

In the case of small molecules, on the other hand, it is expected that after local application they will reach not only the top of a cell layer but also deeper layers of the adjacent tissue. This deep-reaching effect is preferred because tumor cells are also expected there.

A simple form of therapy in the sense of the invention is characterized by the use of at least one type of oligonucleotide as an active ingredient component, which achieves an antitumor effect already individually. This simple form of therapy is preferred in order to maintain a low risk of undesirable effects.

### PREFERRED EMBODIMENTS

A treatment using only one application is preferred, since access to the surgical cavity is limited in time, in view of the rapid development of laparoscopic surgical techniques used in the case of tumor resections, and, ideally, currently does not exceed more than 30 minutes. Each subsequent opening of the surgical cavity would also increase the risk of disseminating tumor cells.

According to the invention, preferably pleiotropic oligonucleotides are used in order to act on as many tumor cells as possible, irrespective of the phase of the cell cycle in which the cell is currently located, in particular in the case of a single application. According to the invention, pleiotropic oligonucleotides are those oligonucleotides which act simultaneously in a cell against at least two targets, for example against two mRNAs having the same target sequence, which mRNAs code for different proteins. Prodrugs according to WO 2010/102615 are an example for a pleiotropic oligonucleotide, wherein preferred embodiments may be effective in tumor cells against several physiological targets in parallel (WO 2012/098234).

According to the invention, preferred is the use of biodegradable carrier materials having low immunogenicity, which exhibit hydrogel-like or elastic properties, and which elute oligonucleotides by diffusion or during in vivo degradation or by changes in parameters of the surroundings such as pH. Carriers, which are medically proven materials such as collagen, atelocollagen, gelatin, fibrin, chitosan or hyaluronic acid, synthetic or recombinant variants thereof and their synthetic modifications, are used with particular preference.

It is particularly preferred if the use of oligonucleotides according to the invention is in carriers which are temporarily associated, such that a large amount of oligonucleotide is available locally and over an extended period of time. According to the invention, “temporarily associated” means that the carrier may be solid, elastic or even deformable, but constitutes a unit over several hours or days. Biodegradable carriers also are considered to be temporarily associated, wherein the carriers become smaller as a result of breakdown processes within the body and which disintegrate into smaller units after hours or days, which are dismantled from a carrier like a gauze, or which undergo a transformation into a liquid phase.

The use of the oligonucleotides according to the invention is particularly preferred in large amounts per application. The total amount of oligonucleotides used depends on the resulting surgical cavity and the structure of the surrounding tissue. For the associated carriers, this association is described as loading density. The use of oligonucleotides is preferred in carriers with loading densities of more than 3 micrograms of oligonucleotide per milliliter of carrier volume, particularly preferably more than 12, 50, 250, 1000 or 5000 micrograms per milliliter, respectively.

The use of carriers may also be advantageous, from which the oligonucleotides elute over an extended period of time, in particular over several days. Carriers that are absorbed by the body are particularly advantageous. Carriers that make possible a simple laparoscopic handling, such as flexible foils or rods, are particularly advantageous. Oligonucleotides and smaller particulate carriers may be applied in nets, such as nets made of gauze material, or in a hydrogel. Preferred is the use of carriers which have an initial elution rate of more than 1 microgram/square centimeter and day, in particular more than 2, 5, 10, 25 or 100 microgram/square centimeter and day, respectively.

According to the invention, the oligonucleotides are used locally after the tumor resection in order to inhibit a recurrence-development or metastases-development. Accordingly, after the complete or partial removal of the primary tumor, they are applied during the surgery to the resulting surgical margins and the surrounding tissue. This also includes surrounding connective and fatty tissues. For a mechanical application of the oligonucleotides, gel-like formulations are advantageous, which may be distributed or sprayed onto the tissue with a brush or similar tool.

Collagen, which may be provided with gel-like or elastic properties, is suitable as a carrier material in the use of the oligonucleotides. Collagen is absorbed by the body and exhibits low immunogenicity.

The use of oligonucleotides in liquid formulation according to the invention is advantageous if the oligonucleotides may be distributed in the surgical cavity using a syringe. An advantage of this form of application is provided in the case of complex shapes of surgical cavities because the formulation distributes itself within the surgical cavity and may flow out of its openings, just as likely as seeding metastases. “Complex” in the sense of the invention means that the surgical cavity is open to cavities such as the peritoneum, through which tumor cells can get into remote margins of the peritoneum.

A particularly suitable distribution is ensured by the use of liquid volumes that are of similar size, particularly preferably of the same size or even 1, 2, 5 times as large as the volume of the surgical cavity alone or including cavities connected thereto. For example, in the case of the removal of an adrenal gland, a surgical cavity of approximately 25 ml is created, which is connected, however, to the much larger retroperitoneal space. With large volumes of liquid, it is possible to also carry out perfusions, such that tumor-effective oligonucleotides are continuously supplied to the surgical cavity and high local concentrations of oligonucleotides may be achieved everywhere in the cavity.

The use of oligonucleotides according to the invention as an aerosol is also preferred. For this purpose, an overpressure is created in the surgical cavity or in the cavities adjacent thereto. The aerosol is introduced into the resulting cavity through a trocar, tube or similar tool or is even generated with an inserted aerosol generator. The aerosol is distributed almost uniformly in the cavity, such that even remote areas of the cavity are reached. The cavity is maintained open for more than two hours, and particularly more than an hour or half an hour. The administration of the oligonucleotides according to the invention in an aerosol form may also be repeated several times if access to the cavity is continuous or may be re-established quickly and easily.

Preferred is the use of oligonucleotides in aerosols in amounts of more than 1 microgram/square centimeter of cavity volume, and particularly preferably of more than 2.5, 10, 25, 100 micrograms.

The use of the oligonucleotides according to the invention includes combinations with methods and therapies known from the prior art. This includes, for example, the simultaneous use of medicaments such as cytostatics and oligonucleotides, and also, for example, a radiotherapy carried out in parallel.

### EXEMPLARY EMBODIMENT

### Adrenocortical Carcinoma

Carcinomas of the adrenal cortex are rare in occurrence and as of today are usually treated by laparoscopic adrenalectomy, i.e., the minimally invasive surgical removal of the complete adrenal gland. Prognosis for patients thus treated nevertheless is poor, particularly in the case of late-identified primary tumors; recurrences or metastases are very likely to occur and usually lead to the patient's death within a few months.

Laparoscopic adrenalectomy is performed using either abdominal/transperitoneal or retroperitoneal access. The renal fascia is pierced and a surgical cavity is created, which is pressurized at 20-30 mm Hg positive pressure for stabilization. The positive pressure also reduces or prevents bleeding into the surgical cavity following any injury to blood vessels. Subsequently, further incisions are used to mobilize surrounding organs, to sever arteries and veins supplying the adrenal gland, and to expose the adrenal gland. Ideally, the adrenal gland as a whole is dissected together with the surrounding adipose tissue, transferred to a retrieval pouch, which in turn is pulled out of the patient via one of the trocars. A lymphadenectomy may be performed in addition. The surgical cavity is rinsed with distilled water and antibiotics and, in simple cases, closed without drainage.

In this surgical procedure, it is carefully avoided that the tumor capsule is damaged and that any tumor tissue remains in the patient. Nevertheless, local recurrences of unclear origin commonly occur. Tumor cells may have been transported from the adrenal gland into the surroundings prior to surgery. This includes seeding metastases occurring in body cavities on other organs or parts of organs caused by caudal migration of detached tumor cells by gravity. These processes are facilitated by the fact that the renal fascia surrounding the kidney opens medially and caudally into the retroperitoneal space.

According to the invention, oligonucleotides are used locally after tumor resection in order to prevent recurrence-development or metastases-development and to fight remaining tumor cells or metastases. To this end, for example, after removal of the adrenal gland, the oligonucleotides are applied during surgery to the resulting surgical margins and the surrounding tissue. This also includes surrounding connective and fatty tissue, especially the renal fascia. Particularly suitable for this purpose are gel-like formulations which may be applied laparoscopically to the wound margins using an instrument such as a brush. In the surgical cavity of an adrenocortical carcinoma, wound margins having a surface area of about 50 square centimeters are created. Gels are applied with a layer thickness of 0.2 to 1 mm, which corresponds to a gel volume of 1 to 5 ml. This gel volume preferably contains 70 micrograms of oligonucleotide and particularly preferably more than 250 or 1000 micrograms, or 5, 25 or 100 milligrams. The gel is degraded within a few weeks and the oligonucleotides are continuously released.

In a further exemplary embodiment, elastic carriers made of collagen, for example, are used, which are introduced into the surgical cavity. As highly elastic carriers, they are introduced into the surgical cavity via the aperture of a trocar as a capsule. After the encapsulation has been removed, the carrier takes on the size and shape corresponding to the resected organ of about 4*3*2 cm, having a volume or partial volume of less than 25 ml. It carries oligonucleotides in amounts of preferably more than 70 micrograms, and particularly preferably more than 250 or 1000 micrograms, or 5, 25 or 100 milligrams. The carrier is degraded within a few weeks and the oligonucleotides are continuously released.

The degradation rate of the gel and the elastic carrier is controllable by manufacturing parameters, in the case of collagen, for example, by the degree of cross-linking of the collagen. In vivo half-lives of more than 1 week are preferred, half-lives of 2 weeks, 4 weeks and 3 months are particularly preferred.

In a further embodiment according to the invention, a liquid formulation for the oligonucleotides is used. Here, the oligonucleotides are distributed in the surgical cavity, for example, by a syringe. An advantage of this form of application is that the formulation distributes itself within the surgical cavity and may flow out of its openings, just as likely as seeding metastases also would do. In adrenocortical carcinoma with the aforementioned expansion of the renal fascia, liquid forms of application reach its extensions much better than solid forms of application. The injected volume in this example is 20 ml, which comprises preferably more than 70 micrograms, and particularly preferred more than 250 or 1000 micrograms, or 5, 25 or 100 milligrams of oligonucleotide. The oligonucleotides may be packed in particles or liposomes such that they are released slowly only when the carriers are degraded or absorbed by the cells. The rate of degradation of the particles may be influenced by manufacturing parameters such as the degree of crosslinking. In vivo half-lives of more than 1 week are preferred, half-lives of 2 weeks, 4 weeks and 3 months are particularly preferred.

It may also be advantageous to use carriers having a simpler geometry, from which the oligonucleotides elute over an extended period, in particular over several days. Carriers which are absorbed by the body are particularly advantageous. Carriers which make possible a simple laparoscopic handling, such as flexible foils or rods, are particularly advantageous. Smaller particulate carriers may be applied in nets, such as gauze nets, or in a hydrogel.

In the case of adrenocortical carcinoma, the use of the oligonucleotides as an aerosol may be preferred, in particular in amounts of 70 micrograms, and particularly preferred in the amount of more than 250 or 1000 micrograms, or 5, 25 or 100 milligrams of oligonucleotide.

The combination of the adjuvant use of the oligonucleotides with adjuvant therapies such as mitotane and the combination with radiotherapy may be preferred.

### EXEMPLARY EMBODIMENT

### Ovarian Cancer

Currently, ovarian cancer is the sixth most common malignant disease in women (Guideline Ovarian Cancer 2013). The surgical removal of an ovary constitutes an essential part of the treatment. In the case of ovarian cancer, the result of the diagnosis strongly influences the extent in which tissue will be removed. In addition to the ovary itself, the fallopian tube and lymph nodes, and, in later stages, also other organs may be affected, in particular those extending into or adjacent to the peritoneum. The tumor resection is carried out as far as possible, but complete removal often is not possible in these cases because the surrounding tissue is already affected. In contrast to adrenocortical carcinoma, it is not recommended to perform the surgery laparoscopically.

Adjuvant chemotherapy is recommended in most cases; an exception to this recommendation relates to cases of very early stages at the time of surgery. Systemic application of carboplatin, a low molecular weight cytostatic, is recommended, which often is associated with significant side effects such as changes in blood count, dysfunction of the liver and nerves, as well as impaired cardiovascular function. In cases of a higher staging level, systemic therapy using paclitaxel and bevacizumab, a monoclonal antibody against VEGF, is also recommended. Relapses nevertheless occur frequently.

According to the invention, the oligonucleotides are used locally after the tumor resection in order to prevent recurrence-development or metastases-development and to fight remaining tumor cells or metastases. To this end, they are applied, for example, during surgery after removal of the ovary and other tissue to the resulting surgical margins and the surrounding tissue. This also includes surrounding connective and fatty tissue, especially parts of the peritoneum.

The size of an ovary in an adult is about 3.5*2*1 cm, its volume amounts to about 3-6 ml. Similar to adrenocortical carcinoma, various formulations are suitable, including a gel-like formulation. Despite the smaller organ volume compared to the adrenal cortex, volumes of 1-5 ml are used preferably since tissue surrounding the ovary should be coated to a greater extent. The gel volume preferably contains 70 micrograms and, particularly preferred, more than 250 or 1000 micrograms, or 5, 25 or 100 milligrams. The gel is degraded within a few weeks and the oligonucleotides are continuously released. Here, the oligonucleotides may be packed in particles or liposomes such that they are released slowly only when the carriers are degraded or are absorbed by the cells. The rate of degradation of the particles may be influenced by manufacturing parameters such as the degree of crosslinking. In vivo half-lives of more than 1 week are preferred, half-lives of 2 weeks, 4 weeks and 3 months are particularly preferred.

In a further exemplary embodiment, elastic carriers made of collagen, for example, are used, which are introduced into the surgical cavity and which completely or partially expand to the volume of an ovary. A carrier carries oligonucleotides in amounts of preferably more than 70 micrograms, and particularly preferred of more than 250 or 1000 micrograms, or 5, 25 or 100 milligrams. The carrier is degraded by the body within a few weeks and the oligonucleotides are continuously released.

The degradation rate of the gel and the elastic carrier are controllable by manufacturing parameters, in the case of collagen, for example, by the degree of cross-linking of the collagen. In vivo half-lives of more than 1 week are preferred, half-lives of 2 weeks, 4 weeks and 3 months are particularly preferred.

In cases of ovarian cancer, a liquid formulation for the oligonucleotides may also be advantageous, since the peritoneum surrounding the ovaries extends wide. The injected volume for ovarian cancer in this example is 20 ml, comprising preferably more than 70 micrograms, and particularly preferred more than 250 or 1000 micrograms, or 5, 25 or 100 milligrams of oligonucleotide. The oligonucleotides may be packed in particles or liposomes such that they are released slowly only when the carriers are degraded or are absorbed by the cells. The rate of degradation of the particles may be influenced by manufacturing parameters such as the degree of crosslinking. In vivo half-lives of more than 1 week are preferred, half-lives of 2 weeks, 4 weeks and 3 months are particularly preferred.

It may also be advantageous to use carriers having a simpler geometry, from which the oligonucleotides elute over an extended period, in particular over several days. Carriers which may be absorbed by the body, such as flexible foils or rods, are particularly advantageous. Smaller particulate carriers may be applied in nets, such as gauze nets, or in a hydrogel.

In the case of ovarian cancer, the use of the oligonucleotides as an aerosol may be preferred, in particular in amounts of 70 micrograms, and particularly preferred in the amount of more than 250 or 1000 micrograms, or 5, 25 or 100 milligrams of oligonucleotide.

The combination of the adjuvant use of the oligonucleotides with adjuvant cytostatics such as carboplatin, cisplatin, paclitaxel and bevacizumab, and the combination with radiation therapy may be preferred.

### EXEMPLARY EMBODIMENT

### Mesothelioma

Malignant diffuse mesothelioma is a tumor originating from the mesothelial or submesothelial cells of the pleura, peritoneum or pericardium. The prognosis for patients with malignant pleural mesothelioma is poor with median survival times of 4 to 12 months. A curative treatment is currently not available.

The predominate amount (>80%) of mesotheliomas originate from the pleura. Malignant mesotheliomas are comparatively rare. They mostly emerge as signal tumors of a previous exposure to asbestos (Neumann et al. 2013). It is also expected that cases of mesothelioma, e.g., after the attack on the World Trade Center in New York on September 11, 2001, will increase significantly in numbers during the next 15 years among those exposed at that time (Povtak 2016). Depending on the subtype of mesothelioma, in more than 50% of pleural mesotheliomas, tumor cells are released into the pleural effusion.

There is no standard therapy for treating mesothelioma. Recommended therapy concepts range from standalone symptomatic treatment to aggressive multimodal treatment comprising surgery, chemotherapy and radiation. Currently, two surgical strategies exist, namely, pleurectomy/decortication or extrapleural pleuropneumectomy, in order to achieve as complete a macroscopic tumor removal as possible. These resections are not performed laparoscopically. Due to the diffuse growth of the mesothelioma, however, a complete tumor removal generally is not possible. Residual parts of the tumor remain, which often are detectable only by microscope, in the case of which adjuvant radiotherapy and chemotherapy are recommended (Rice 2011). According to the prior art, a combination of intraperitoneal carboplatin and pemetrexed is recommended. Regarding patient survival, successes owing to the adjuvant use of check point inhibitors were recently noted (Scherpereel et al. 2017).

In contrast to carcinomas of the adrenal cortex and ovary, the size of the surgical cavity differs widely; after resection of a lung in pleural mesothelioma, it may amount to a size of several liters with an area of several hundred square centimeters. In the case of these volumes, a gel may also be used advantageously and applied with tools such as brushes; concentrations of more than 15 or 70 micrograms per milliliter of gel are preferred, particularly preferred of more than 250 or 1000 micrograms, or 5, 25 or 100 milligrams per milliliter of gel.

In the case of retaining the affected lung, a liquid formulation is suitable, preferably at concentrations of more than 15 or 70 micrograms per milliliter and particularly preferred at more than 250 or 1000 micrograms, or 5, 25 or 100 milligrams per milliliter.

In the case of large-volume resections, elastic carriers having a total volume corresponding to that of the resected organs are no longer useful. Instead, it is better that flat carriers, such as membranes, foils or gauze, are used, which carry oligonucleotides directly, packaged in particles such as liposomes, conjugated or formulated as a gel. Loading densities of 10 micrograms per square centimeter are preferred, particularly preferred are densities of 50 or 200 micrograms, or 1, 5 or 20 milligrams of oligonucleotide per square centimeter. Preferred is the application of oligonucleotides onto implants, such as those used after resection of the diaphragm, with the same oligonucleotide density.

Also in the case of mesothelioma, the use of carriers having a simpler geometry may be preferred, in particular if the oligonucleotides elute from these carriers over a longer period, in particular over several days. Carriers that are absorbed by the body, such as flexible foils or rods, are particularly advantageous. Smaller particulate carriers can be applied in nets, such as gauze nets, or in a hydrogel.

In mesothelioma, the use of the oligonucleotides as an aerosol may be preferred, in particular in amounts of 70 micrograms, and particularly preferred in the amount of more than 250 or 1000 micrograms, or 5, 25 or 100 milligrams of oligonucleotide.

The combination of the adjuvant use of the oligonucleotides with further adjuvant therapies such as carboplatin and pemetrexed, and the combination with radiotherapy may be preferred.

### EXEMPLARY EMBODIMENT

### Glioblastoma

Glioblastomas belong to the diffusely infiltrating, highly malignant gliomas and are the most common brain neoplasms with a share of 16%. According to the WHO classification, they are classified as grade IV tumors and are associated with a poor prognosis. Since glioblastomas exhibit a markedly infiltrative growth, a cure by surgical resection of the tumor is not possible. It is a goal to reduce the tumor mass surgically as completely as possible. An adjuvant therapy, in which radiotherapy is combined with chemotherapy, is therefore recommended according to the European Organization for Research and Treatment of Cancer and National Cancer Institute of Canada Clinical Trials Group (EORTC-NCIC) protocol (Javamanne et al., 2018). It commences the earliest at four weeks after the operation, however, when the surgical wound healing process has progressed. Currently, the treatment standard is temozolomide (Davis 2016), an oral alkylating chemotherapy drug, which is however genotoxic and teratogenic. The above-described adjuvant deposition of Gliadel™ wafers during the surgery is a treatment variant that is not part of the standard of treatment, but which also is carried out together with temozolomide treatment. In contrast, the simultaneous use of temozolomide and bevavizumab is not recommended (Holdhoff et al. 2011). A newer experimental treatment option includes DCVax, a procedure in which monocytes are removed from the patient and differentiated extracorporeally into dendritic cells carrying tumor antigens, and which are subsequently transferred back into the patient.

In glioblastoma, the size and shape of the surgical cavity differ individually. The size of the surgical cavity, however, also amounts to a few milliliters, making it comparable to that of adrenocortical carcinoma and ovarian carcinoma. The shape is different, however, and as individual as the size. In addition, partial resections are performed almost exclusively in the case of glioblastoma.

For the use according to the invention in the case of glioblastoma, gel-like formulations, which are applied to the wound margins using an instrument such as a brush, are also particularly suitable. The gel volume of 1 to 5 milliliters is preferred to contain 70 micrograms, particularly preferred more than 250 or 1000 micrograms, or 5, 25 or 100 milligrams. The gel is degraded within a few weeks and the oligonucleotides are continuously released. The oligonucleotides may be packed in particles or liposomes such that they are released slowly only when the carriers are degraded or are absorbed by the cells. The rate of degradation of the particles may be influenced by manufacturing parameters such as the degree of crosslinking. In vivo half-lives of more than 1 week are preferred, half-lives of 2 weeks, 4 weeks, 3 months or an entire year are particularly preferred.

In a further exemplary embodiment, elastic carriers made of collagen, for example, are used, which are introduced into the surgical cavity and which completely or partially expand to the volume of the surgical cavity. The one or the carriers carry oligonucleotides in amounts of preferably more than 70 micrograms, particularly preferred of more than 250 or 1000 micrograms, or 5, 25 or 100 milligrams. The one or the carriers are degraded by the body within a few weeks and the oligonucleotides are continuously released.

The degradation rate of a gel or of an elastic carrier is controllable by manufacturing parameters, in the case of collagen, for example, by the degree of cross-linking of the collagen. In vivo half-lives of more than 1 week are preferred, half-lives of 2 weeks, 4 weeks and 3 months are particularly preferred.

In cases of glioblastoma, a liquid formulation for the oligonucleotides may also be advantageous, since it is not known whether the glioblastoma also spreads in a manner other than infiltrating. In the case of glioblastoma, the injected volume, for example, amounts to 20 ml, in which preferably more than 70 micrograms, particularly preferred more than 250 or 1000 micrograms, or 5, 25 or 100 milligrams of oligonucleotide is found. The oligonucleotides may be packed in particles or liposomes such that they are released slowly only when the carriers are degraded or are absorbed by the cells. The rate of degradation of the particles may be influenced by manufacturing parameters such as the degree of crosslinking. In vivo half-lives of more than 1 week are preferred, half-lives of 2 weeks, 4 weeks and 3 months are particularly preferred.

In the case of glioblastoma, the use of the oligonucleotides as an aerosol may be preferred, in particular in amounts of 70 micrograms, and particularly preferred in the amount of more than 250 or 1000 micrograms, or 5, 25 or 100 milligrams of oligonucleotide

It may also be advantageous to use carriers having a simpler geometry, from which the oligonucleotides elute over an extended period, in particular over several days.

Carriers which are absorbed by the body, such as flexible foils or rods, are particularly advantageous. Smaller particulate carriers may be applied in nets, such as gauze nets, or in a hydrogel. Another example are wafers as used in Gliadel.

The combination of the adjuvant use of the oligonucleotides with adjuvant cytostatics such as temozolomide, with cellular therapies such as DCVax, and the combination with radiation may be preferred.

### APPLICATION EXAMPLE

### Peritoneal Carcinosis

Peritoneal carcinosis refers to the infestation of the peritoneum with multiple malignant tumor cells. The cause of a peritoneal carcinosis is usually not a tumor of the peritoneum itself, but rather a malignant tumor of another organ located in the abdomen. Most commonly, this is an advanced metastatic tumor of the gastrointestinal tract, of the pancreas or of the ovaries, as described above. In some cases, it is not possible to identify a primary tumor. (https://flexikon.doccheck.com/de/Peritonealkarzinose),

At 15-20 cases per 100,000 people, peritoneal carcinosis occurs rarely, but with an increasing tendency; in Germany there are about 35,000 new cases per year (Glockzin et al. 2007). The prognosis of the peritoneal carcinosis is on average about 6 months after diagnosis.

As long as it has not advanced too far, peritoneal cancer is being treated increasingly using multimodal therapy in which surgical cytoreduction is combined with an intraoperative, hyperthermal intraperitoneal chemotherapy (Piso et al. 2011). PIPAC, as described above, also is used.

In contrast to the adrenocortical and ovarian carcinomas, the size of the surgical cavity differs greatly individually due to the varying number of affected organs. A gel can nevertheless be advantageously used for these volumes and can be applied with tools such as brushes; concentrations of more than 15 or 70 micrograms per milliliter of gel are preferred, particularly preferred are concentrations of more than 250 or 1000 micrograms, or 5, 25 or 100 milligrams per milliliter of gel.

In the case of large-volume resections, elastic carriers having a total volume corresponding to that of the resected organs are no longer useful. Instead, it is better that flat carriers, such as membranes, foils or gauze, are used, which carry oligonucleotides directly, packaged in particles such as liposomes, conjugated or formulated as a gel. Loading densities of 10 micrograms per square centimeter are preferred, particularly preferred are densities of 50 or 200 micrograms, or 1, 5 or 20 milligrams of oligonucleotide per square centimeter. Preferred is the application of oligonucleotides onto implants, such as those used after resection of the diaphragm, with the same oligonucleotide density.

Also in the case of peritoneal carcinosis, the use of carriers having a simpler geometry may be preferred, in particular if the oligonucleotides elute from these carriers over a longer period, in particular over several days. Carriers that are absorbed by the body, such as flexible foils or rods, are particularly advantageous. Smaller particulate carriers can be applied in nets, such as gauze nets, or in a hydrogel.

In peritoneal carcinosis, the use of the oligonucleotides as an aerosol may be preferred, in particular in amounts of 70 micrograms, and particularly preferred in the amount of more than 250 or 1000 micrograms, or 5, 25 or 100 milligrams of oligonucleotide.

The combination of the adjuvant use of the oligonucleotides with adjuvant therapies such as carboplatin and pemetrexed, and the combination with radiotherapy may be preferred.

### CITATIONS

Berrada M et al, “A novel non-toxic camptothecin formulation for cancer chemotherapy”, Biomaterials. 2005 May; 26 (14): 2115-20.
Blanco E et al, “Local release of dexamethasone from polymer millirods effectively prevents fibrosis after radiofrequency ablation”, J Biomed Mater Res A. 2006 January; 76 (1): 174-82.
de Boer, E. (2016). Antibody Based Surgical Imaging and Photodynamic Therapy for Cancer [Groningen]: Rijksuniversiteit Groningen
Ceelen W et al “Intraperitoneal therapy for peritoneal tumors: biophysics and clinical evidence” Nat Rev Clin Oncol 2010; 7: 108-115.
Chowdhary et al., “Survival outcomes and safety of carmustine wafers in the treatment of high-grade gliomas: a meta-analysis”, J Neurooncol. 2015; 122 (2): 367-382, doi: 10.1007/s11060-015-1724-2
Colson Y L et al, “The performance of expansile nanopartides in a murine model of peritoneal carcinomatosis” biomaterials. 2011 January; 32 (3): 832-40.
Davis M E “Glioblastoma: Overview of Disease and Treatment” Clin J Oncol Nurs. 2016 Oct 1; 20 (5): S2-S8. doi: [10.1188/16.CJON.S1.2-8]
De Gramont A et al., “From Chemotherapy to Targeted Therapy in Adjuvant Treatment for Stage III Colon Cancer”, Seminars in Oncology, Vol 38, No 4, August 2011, pp 521-532
Du Y et al, “Improved resection and prolonged overall survival with PD-1-IRDye800CW fluorescence probe-guided surgery and PD-1 adjuvant immunotherapy in 4T1 mouse model”, Int. J. of Nanomedicine Volume 2017: 12 8337-8351, DOI https://doi.org/10.2147/IJN.S149235
Glehen O et al “Hyperthermia Intraperitoneal Chemotherapy: Nomenclature and Modalities of Perfusion” J. Surg. Oncol. 2008; 98: 242-246. © 2008 Wiley-Liss, Inc. https://doi.org/10.1002/jso.21061
Glockzin et al “Peritoneal Cancer Surgical Treatment Options including hyperthermic intraperitoneal chemotherapy”, surgeon 2007 DOI 10.1007/S00104-007-1419-0
Griset A P et al, “Expansile nanopartides: synthesis, characterization, and in vivo efficacy of an acid-responsive polymeric drug delivery system” J Am Chem Soc. 2009 Feb. 25; 131 (7): 2469-71.
Haaga J R, “Combined tumor therapy by using radiofrequency ablation and 5-FU-laden polymer implants: Haaga JRevaluation in rats and rabbits” Radiology. 2005 December; 237 (3): 911-8.
Han H D et al “Chitosan hydrogel for localized gene silencing”, Cancer Biol Ther. 2011 May 11 (9): 839-45
Hart M G. et al., “Chemotherapeutic wafers for high grade glioma” Cochrane Database Syst Rev. 2008 Jul 16; (3): CD007294. doi: 10.1002/14651858.CD007294.
Huang L et al “CpG-based immunotherapy impairs antitumor activity of BRAF Inhibitors in a B-cell-dependent manner”, Oncogene volume 36, pages 4081-4086 (13 Jul. 2017)
Herdewijn P “Oligonucleotide Synthesis: Methods and Applications”, Springer Science & Business Media, 2005, ISBN 1592598234, 9781592598236
Holdhoff M et al “Controversies in the Adjuvant Therapy of High-Grade Gliomas” The Oncologist March 2011 vol. 16 no. 3 351-358, doi: 10.1634/theoncologist.2010-0335
Houot R et al “T-cell modulation combined with intratumoral CpG your lymphoma in a mouse model without the need for chemotherapy” BLOOD, 9 APR. 2009 Vol 113, No 15 pp 3546-3552
Javamanne D et al “Survival improvements with adjuvant therapy in patients with glioblastoma” ANZ J Surg. 2018 Mar; 88 (3): 196-201. Doi: 10.1111/ans.14153. Epub 2017 Sep. 18.
Khairuddin N et al “In vivo comparison of local versus systemic delivery of immunostimulating siRNA in HPV-driven tumors”, Immune and Cell Biology (2014) 92, 156-163 doi: 10.1038/icb.2013.75
Khvalevsky E Z et al “Mutant KRAS is a druggable target for pancreatic cancer”, Proc Natl Acad Sei U S A. 2013 Dec. 17; 110 (51): 20723-20728 doi: 10.1073/pnas.1314307110
Kim I Y et al, “Chitosan and its derivatives for tissue engineering applications”, Biotechnol Adv. 2008 January-February; 26 (1): 1-21.
Kim Y M et al “Injectable polyplex hydrogel for localized and long-term delivery of siRNA”, ACS Nano. 2012 Jul. 24; 6 (7): 5757-66. doi: 10.1021/nn300842a. Epub 2012 Jun. 8
Korb ML et al, “Use of monoclonal antibody-IRDye800CW bioconjugates in the resection of breast cancer”, J Surg Res. 2014 May 1; 188 (1): 119-28. doi: 10.1016/j.jss.2013.11.1089. Epub 2013 Nov. 22
Krebs M D et al, “Localized and sustained delivery of silencing RNA from macroscopic biopolymer hydrogels”, J Am Chem Soc. 2009 Jul. 8; 131 (26): 9204-6. doi: 10.1021/ja9037615
Guideline for Ovarian Cancer 2013 “Guideline Program Oncology” (German Cancer Society, German Cancer Aid, AWMF): S3 Guideline Diagnostics, therapy and aftercare of malignant ovarian tumors, long version 1.0, AWMF registration number: 032-035OL, https/leitlinienprogramm-onkologie.de/Leitlinien.7.0.html
Li K W et al, “Polilactofate microspheres for Paclitaxel delivery to central nervous system malignancies”, Clin. Cancer Res. 9 (2003) 3441-3447
Liu R et al, “Prevention of local tumor recurrence following surgery using low-dose chemotherapeutic polymer films” Ann Surg Oncol. 2010 April; 17 (4): 1203-13
Liu R et al, “Paclitaxel-eluting polymer film reduces locoregional recurrence and improves survival in a recurrent sarcoma model: a novel investigational therapy” Ann Surg Oncol. 2012 January; 19 (1): 199-206.
Marabelle A et al “Intratumoral immunotherapy: using the tumor as the remedy”, Annals of Oncology 28 (Supplement 12): xii33-xii43, 2017 doi:10.1093/
Nelson C E et al, “Tunable Delivery of siRNA from a Biodegradable Scaffold to Promote Angiogenesis In vivo” Adv Mater. 2014 January; 26 (4): 607-506, Published online 2013 Dec. 16. doi: 10.1002/adma.201303520
Neumann V, Loseke S, Nowak D, Herth F J F, Tannapfel A “Malignant pleural—mesothelioma—incidence, etiology, diagnosis, treatment and occupational health”. Dtsch Arztebl Int 2013; 110 (18): 319-26. DOI: 10.3238/arzteb1.2013.0319
Nguyen PhD et al “Improved diabetic wound healing through topical silencing of p53 is associated with augmented vasculogenic mediators” Wound Repair Regen. 2010 November-December; 18 (6): 553-559 doi: 10.1111/j.1524-475X.2010.00638.x
Oyan B “Why do targeted agents not work in the adjuvant setting in colon cancer?” Expert Rev. Anticancer Ther. 12 (10), 1337-1345 (2012)
Piso et al. “Multimodal therapy concepts for peritoneal cancer in colorectal cancer”, Dtsch Arztebl Int 2011; 108 (47): 802-8; DOI: 10.3238/arzteb1.2011.0802
Povtak, T. “15th Anniversary of 9/11: Mesothelioma Expected to Rise from Attack” (Interview with Raja Flores, Mt. Sinai) url: https://www.asbestos.com/news/2016/09/08/15-anniversary-9-11-mesothelioma-world-trade-center-attack/
Qian F et al, “Quantification of in vivo doxorubicin transport from PLGA millirods in thermoablated rat livers”, J Control Release. 2003 Aug. 28; 91 (1-2): 157-66
Reymond M et al “Pressure aerosol chemotherapy (PIPAC): less is sometimes more” Passion Surgery Sep. 2014
Rice, D. “Surgical Therapy of Mesothelioma”, in A. Tannapfel (ed.), Malignant Mesothelioma, Recent Results in Cancer Research 189, 97 DOI: 10.1007/978-3-642-10862-4_7, Springer-Verlag Berlin Heidelberg 2011
Rosenblum D et al, “Progress and challenges towards targeted delivery of cancer therapeutics”, Nature Communications v9, Article number: 1410 (2018), doi: 10.1038/541467-018-03705-y
Ruel-Gariépy E et al, “A thermosensitive chitosan-based hydrogel for the local delivery of paclitaxel”, Eur J Pharm Biopharm. 2004 January; 57 (1): 53-63.
Scherpereel A et al, “Second- or third-line nivolumab (Nivo) versus nivo plus ipilimumab (Ipi) in malignant pleural mesothelioma (MPM) patients: Results of the IFCT-1501 MAPS2 randomized phase II trial” 2017 ASCO Annual Meeting, J Clin Oncol 35, 2017 (suppl; abstr LBA8507)
Schulz M D et al, “Paclitaxel-loaded expansile nanoparticles in a multimodal treatment model of malignant mesothelioma” Ann Thorac Surg. 2011 December; 92 (6): 2007-13; discussion 2013-4.
Strong L E et al “Hydrogel-nanoparticle composites for optically modulated cancer therapeutic delivery”, J Control Release. 2014 Mar. 28; 178: 63-8. doi: 10.1016
Surhone et al “Oligonucleotide Synthesis: Solid-Phase Synthesis, DNA, DNA Sequencing, RNA, Small Interfering RNA, Nucleoside, Nucleic Acid, Nudeotide, Phosphoramidite, Sense”, Betascript Publishing, 2010, ISBN 6130300298, 9786130300296
Tokatlian T et al “Non-Viral DNA Delivery from Porous Hyaluronic Acid Hydrogels in Mice” Biomaterials. 2014 January; 35(2): 825-835 doi: 10.1016/j.biomaterials.2013.10.014
Vandegrift M T et al 2015 “Acellular dermal matrix-based gene therapy augments graft incorporation” J Surg Res. 2015 May 1; 195 (1): 360-7. doi: 10.1016
Weinberg B D et al, “Antitumor efficacy and local distribution of doxorubicin via intratumoral delivery from polymer millirods”, J Biomed Mater Res A. 2007 April; 81 (1): 161-70.
Wolinsky J B et al, “Local Drug Delivery Strategies for Cancer Treatment: Gels, Nanoparticles, Polymeric Films, Rods, and Wafers” J Control Release. 2012 Apr. 10; 159 (1): 10.1016/j.jconrel.2011.11.031.
Wolinsky J B et al, “Prevention of in vivo lung tumor growth by prolonged local delivery of hydroxycamptothecin using poly (ester-carbonate)-collagen composites” Control Release. 2010 Jun. 15; 144 (3): 280-7
Yang J et al, “Development of bioactive materials for glioblastoma therapy”, Bioactive Materials Vol 1, Issue 1, September 2016, 29-38

1. A method of treating a patient having a solid tumor, comprising:
performing a surgical procedure and creating an incision in the patient to perform complete or partial resection or ablation of the solid tumor, and
then, before completing the surgical procedure and closing the incision or before removing a drainage inserted during the surgical procedure, locally applying a therapeutically-effective amount of an oligonucleotide that kills tumor cells in a body cavity created by the resection or ablation in order to fight tumor cells remaining in, or in the surroundings of, a tumor bed and/or to fight metastases, the oligonucleotide counteracting development of recurrences of the solid tumor or new metastases.

2. The method according to claim 1, wherein the oligonucleotide acts in a pleiotropic manner in at least one type of tumor cell.

3. The method according to claim 1, wherein the oligonucleotide is applied in a gel, in a liquid or in an elastic material.

4. The method according to claim 1, wherein the oligonucleotide is applied as a load of a carrier in the form of a gel, a liquid or an elastic material, and wherein the carrier contains the oligonucleotide at a concentration of more than 3 micrograms per milliliter.

5. The method according to claim 4, wherein the carrier comprises a resorbable elastic or gel material and at least partially reproduces the shape of the resected solid tumor.

6. The method according to claim 1, wherein the oligonucleotide is applied as a load of a flat carrier composed of a membrane, a foil or a gauze, wherein the oligonucleotide is carried in the flat carrier directly, packaged in particles, conjugated thereto or formulated as a gel, and wherein that the oligonucleotide is present in the flat carrier in a loading density of at least 10 micrograms per square centimeter.

7. The method according to claim 4, wherein the carrier contains collagen, atelocollagen, gelatin, chitosan or hyaluronic acid.

8. The method according to claim 6, wherein the carrier additionally contains one or more absorptive or fluorescent dyes.

9. The method according to claim 4, wherein the carrier comprises a crosslinked or otherwise stabilized material that degrades in the patient's body at an in vivo half-life of more than a week.

10. The method according to claim 4, wherein the carrier further comprises a therapeutically-effective amount of a chemotherapy medicament.

11. The method according to claim 4, wherein the carrier is composed of two or more of the gel, the gauze and the liquid to optimally reach tumor cells which are located at a distance from a wound margin created by the resection or ablation.

12. The method according to claim 4, further comprising one or more diagnostic elements integrated on the carrier.

13. The method according to claim 1, further comprising carrying out at least one standard therapy selected from aftercare and adjuvant chemotherapy in parallel with or almost simultaneously with the the steps of tumor resection or ablation and oligonucleotide application.

14. The method according to claim 1, wherein the method is for adjuvant therapy of adrenocortical carcinoma, ovarian cancer, mesothelioma or glioblastoma.

15. The method according to claim 1, wherein the oligonucleotide is applied locally to the tumor bed or to a surgical margin in a carrier that has an initial elution rate of more than 1 microgram/square centimeter per day.

16. The method according to claim 1, wherein the oligonucleotide is applied laparoscopically to the tumor bed or to a surgical margin using a brush.

17. The method according to claim 1, wherein the oligonucleotide is applied as an aerosol to the tumor bed or to a surgical margin using a brush.
